(19) United States
(12) Patent Application Publication
(10) Pub. No.: US 2014/0171682 A1
(43) Pub. Date: Jun. 19, 2014

(54) Creatine hydrochloride and manufacturing method thereof

(71) Applicant: Guoji Zhang, Yangliuqing (CN)

(72) Inventor: Guoji Zhang, Yangliuqing (CN)

(73) Assignee: Tianjin Tiancheng Pharmaceutical Co., Ltd (China)

(21) Appl. No.: 13/694,590

(22) Filed: Dec. 14, 2012

(51) Int. Cl. C07C 277/08

(52) U.S. Cl. CPC C07C 277/08
USPC 562/560

(57) ABSTRACT

A manufacturing method for preparing creatine hydrochlorides includes the steps of using absolute ethyl alcohol as the cleaning agent to reduce production costs and to avoid harm to the human body resulting in the production process.

### NOTICE OF COPYRIGHT

A portion of the disclosure of this patent document contains material which is subject to copyright protection. The copyright owner has no objection to any reproduction by anyone of the patent disclosure, as it appears in the United States Patent and Trademark Office patent files or records, but otherwise reserves all copyright rights whatsoever.

### BACKGROUND OF THE PRESENT INVENTION

1. Field of Invention

The present invention relates to creatine hydrochloride, and more particularly to a manufacturing method for preparing creatine hydrochlorides, wherein the absolute ethyl alcohol is employed as the cleaning agent for cleaning raw crystallized creatine hydrochlorides in the manufacturing method to reduce product costs of creatine hydrochlorides and to avoid damage of the acetone in the conventional manufacturing method.

2. Description of Related Arts

Creatine (known as N-(Aminoiminomethyl)-N-methyl-glycine) is a nitrogenous organic acid present in the heart, the retina, the brain, muscles, and other organs. Creatine is naturally synthesized from L-arginine, glycine, and L-methionine in the kidney, pancreas, and liver. It is transported through the blood and taken up by tissues with high energy demands, such as the brain and skeletal muscles, through an active transport system. Creatine is mainly excreted via the kidneys after it is irreversibly converted into creatinine by skeletal muscles. Creatine plays an important role in the metabolic system. Phosphocreatine is able to help increase the formation of adenosine triphosphate (ATP) in muscles quickly so as to supply ATP for rapid energy consumption during high intensity exercise and to increase muscle mass in the human body. The human body consumes energy when in movement, and the consumed energy comes from this ATP. The consumption of ATP by the human body is provided by the aerobic metabolic pathways in human cells when people do low-intensity exercises. The aerobic exercise depends on the aerobic energy-generating process. The human body consumes a large amount of ATP in a short time when people do high-intensity exercises, and the ATP from the aerobic metabolic cannot meet the energy requirement of this strenuous exercise. At this time, the phosphorylated form of creatine, creatine phosphate (CP), is activated and involved in energy metabolism which quickly degrades and provides ATP to supplement the consumption of ATP during strenuous exercise. As an important energy storage substance, creatine can help to store energy for doing exercises, and effectively increasing muscle mass, strength, and the endurance of the human body to improve athletic performance. In addition, creatine can also automatically adjust the water in muscles and expand the cross-sectional area of muscles to improve the muscle's explosive force.

Furthermore, creatine is widely served in the field of food and drug. Creatine is used as an adaptive agent to promote skeletal muscles to adapt to strenuous exercise and as a nutritional supplement to eliminate the condition of excessive fatigue is a weak body. Creatine can also be used to prepare medicines for the treatment of heart disease and respiratory insufficiency and in pharmaceutical preparations containing one or more human growth hormones. Creatine is also used for formulating a variety of health foods and food additives such as creatine citrate, creatine malic acid, and creatine orotic acid. Since creatine is not a hormone, it does not cause any interference or destruction to the endocrine system of the human body and it does not result in drug dependence or other physiological side effects. A diet related to creatine or as a nutrition supplement is greatly welcomed in the field of sports and health.

Creatine is commonly modified and converted into a water-soluble substance for oral administration, due to the fact that creatine has a characteristic of low water solubility and permeability. Creatine can react with chlorohydric acid to provide creatine hydrochloride for a user, which is a water soluble substance and convenient to absorb.

A conventional manufacturing method for creatine hydrochloride comprises a step of preparing an aqueous solution of the creatine and a step of adding the aqueous solution into a solution of the hydrochloric acid drop by drop. The conventional manufacturing method for making creatine hydrochlorides has several drawbacks. The excessive water content in the reaction solution results in a great deal of the by-product creatinine and reduces the productivity of the creatine hydrochloride. Secondly, the reaction time is lengthened in the conventional manufacturing method such that the time cost for making the creatine hydrochloride is increased. Additionally, the conventional method uses acetone as the cleaning agent for the creatine hydrochloride which is expensive and poisonous to the human body such that the conventional manufacturing method for creatine hydrochloride results in a high preparation cost and creates hazardous working conditions.

### SUMMARY OF THE PRESENT INVENTION

The invention is advantageous in that it provides a method for preparing creatine hydrochlorides, wherein the manufacturing method can provide a high product recovery and product quality.

Another advantage of the invention is to provide a method for preparing creatine hydrochlorides, wherein the manufacturing method does not need additional water as a cosolvent, so as to reduce byproduct of creatinine and improve the productivity of the final products of creatine hydrochlorides.

Another advantage of the invention is to provide a method for preparing creatine hydrochlorides, wherein the manufacturing method allows all of the reaction materials are added into the reaction container at the same time to shorten the production cycle thereof.

Another advantage of the invention is to provide a method for preparing creatine hydrochlorides, wherein the manufacturing method employs alcohol as a cleaning agent for the final product to reduce manufacturing costs, decrease harm to the human body and improve the production environment.

Additional advantages and features of the invention will become apparent from the description which follows, and may be realized by means of the instrumentalities and combinations particular point out in the appended claims.

According to the present invention, the foregoing and other objects and advantages are attained by the manufacturing method comprising the steps of:

(A) adding 5 units of creatine monohydrate and 4 units of 30% hydrochloric acid into a container and stirring until all of creatine monohydrate is dissolved;

(B) adjusting the value of pH to 1.0 by hydrochloric acid;

(C) concentrating, crystallizing, and separating the product of the crystal; and

(D) flushing the achieved crystal by absolute ethyl alcohol.

Still further objects and advantages will become apparent from a consideration of the ensuing description and drawings.

These and other objectives, features, and advantages of the present invention will become apparent from the following detailed description, the accompanying drawings, and the appended claims.

### BRIEF DESCRIPTION OF THE DRAWINGS

FIG. 1 is a flow chart of the manufacturing method according to the preferred embodiment of the present invention.

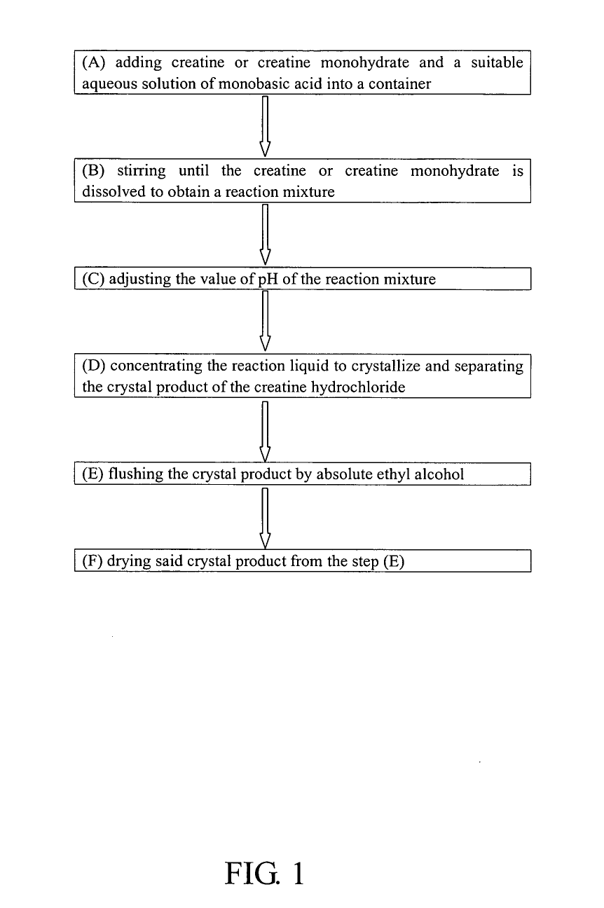

### DETAILED DESCRIPTION OF THE PREFERRED EMBODIMENT

The following description is disclosed to enable any person skilled in the art to make and use the present invention. Preferable embodiments are provided in the following description only as examples and modifications will be apparent to those skilled in the art. The general principles defined in the following description would be applied to other embodiments, alternatives, modifications, equivalents, and applications without departing from the spirit and scope of the present invention.

The following description is disclosed to enable any person skilled in the art to make and use the present invention. Preferable embodiments are provided in the following description only as examples and modifications will be apparent to those skilled in the art. The general principles defined in the following description would be applied to other embodiments, alternatives, modifications, equivalents, and applications without departing from the spirit and scope of the present invention.

According to the preferred embodiment of the present invention, a manufacturing method for preparing various creatine hydrochlorides having characteristics of high solubility and stability is illustrated. Each of creatine hydrochloride molecules includes a molecule of creatine and a molecule of monatomic acid. The suitable hydrochloric acid is selected from the group consisting of food acceptable monatomic inorganic acids, such as hydrochloric acid, and food-acceptable monatomic organic acids, such as acetic acid. These creatine hydrochlorides are generally prepared by reacting creatine with a hydrochloric acid in the aqueous solution to form the corresponding reaction liquid, concentrating the reaction liquid to crystallize, and filtering concentrate to obtain crystal product.

According to the preferred embodiment of the present invention, a creatine hydrochloride is generated by reacting creatine monohydrate with a corresponding hydrochloric acid in a suitable solution until the corresponding creatine hydrochloride is created, and then cooling the reaction liquid and filtering to receive the corresponding creatine hydrochloride. The remaining filtrate can be filtered again or used for further reaction. Any hydrochloric acids can be used in the manufacturing method for making creatine hydrochlorides, wherein the hydrochloric acids include food-grade mineral acids, such as hydrochloric acid, and food-grade organic acids, such as acetic acid. The food-grade creatine or creatine monohydrate is used as another reactant to prepare the corresponding creatine hydrochloride.

According to the preferred embodiment of the present invention, the creatine hydrochloride prepared by utilizing a hydrochloric acid and creatine or creatine monohydrate has the formula of:

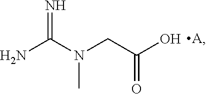

wherein A represents a hydrochloric acid anion. The anion can be a hydrochloric acid anion, acetate anion or other hydrochloric acid anion.

According to the preferred embodiment of the present invention, the manufacturing method for making creatine hydrochlorides comprises the steps of:

(A) adding creatine or creatine monohydrate and the suitable aqueous solution of hydrochloric acid into a container, wherein the ratio of creatine or creatine monohydrate VS the hydrochloric acid in the aqueous solution is 1:1;

(B) stirring until all the creatine or creatine monohydrate is dissolved;

(C) adjusting the value of pH to 1.0 by hydrochloric acid;

(D) concentrating the reaction liquid to crystallize and separating the crystal product to obtain the corresponding raw creatine hydrochloride; and

(E) flushing the crystal product by absolute ethyl alcohol.

The manufacturing method further comprises a step of:

(F) drying the achieved crystal product of the corresponding creatine hydrochloride.

Wherein the concentration of hydrochloric acid in the step A) is 25-35%, preferably 30% ; the reaction temperature in the step (B) is 25° C.; the start time for concentrating to crystallize in the step D) is 30 minutes after the value of pH is adjusted to 1.0; the reaction conditions for concentrating the reaction liquid to crystallize in the step D) is the degree of vacuum of 0.08-0.10 Mpa, preferably 0.09 Mpa, a temperature of 40° C. to 55° C., preferably 50° C.; the amount of anhydrous ethanol used in the step E) is not less than 0.1 times of the weight of the crystal product of the corresponding creatine hydrochloride; the drying temperature in the step F) is 50° C. to 60° C.

The final product generated by the manufacturing method is a creatine hydrochloride, which includes a molecule of creatine and a molecule of monatomic acid anion, wherein the creatine hydrochloride can be the creatine hydrochloride salt, the creatine acetate or other similar creatine maleate.

One skilled in the art will understand that the embodiment of the present invention as shown in the drawings and described above is exemplary only and not intended to be limiting.

It will thus be seen that the objects of the present invention have been fully and effectively accomplished. It embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A manufacturing method for preparing a creatine hydrochloride, comprising the steps of:
(A) adding a creatine and a suitable aqueous solution of hydrochloric acid into a container, wherein the ratio of said creatine VS said hydrochloric acid in said suitable aqueous solution is 1:1;
(B) stirring until all said creatine is dissolved to obtain a reaction mixture;
(C) adjusting the value of pH of said reaction mixture to 1.0 by hydrochloric acid;
(D) concentrating the reaction liquid to crystallize and separating the crystal product of said creatine hydrochloride from said reaction mixture; and
(E) flushing said crystal product by absolute ethyl alcohol;
wherein the formula of said creatine hydrochloride is 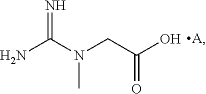
wherein A represents a hydrochloric acid anion.

2. The manufacturing method, as recited in claim 1, further comprising a step (F) of drying said crystal product from the step (E).

3. The manufacturing method, as recited in claim 2, wherein said hydrochloric acid in the step (A) is hydrochloride acid and said creatine hydrochloride is creatine hydrochloride.

4. The manufacturing method, as recited in claim 2, wherein said hydrochloric acid in the step (A) is acetate acid and said creatine hydrochloride is creatine acetate.

5. The manufacturing method, as recited in claim 3, wherein the concentration of said hydrochloride acid is 25-35%.

6. The manufacturing method, as recited in claim 5, wherein the concentration of said hydrochloride acid is 30%.

7. The manufacturing method, as recited in claim 2, wherein the reaction temperature in the step (B) is 25° C.

8. The manufacturing method, as recited in claim 6, wherein the reaction temperature in the step (B) is 25° C.

9. The manufacturing method, as recited in claim 1, wherein the reaction conditions of vacuum level and temperature in the step (D) are 0.08-0.10 Mp and 40° C.-55° C. respectively.

10. The manufacturing method, as recited in claim 8, wherein the reaction conditions of vacuum level and temperature in the step (D) are 0.08-0.10 Mp and 40° C.-55° C. respectively.

11. The manufacturing method, as recited in claim 9, wherein said reaction conditions of vacuum level and temperature in the step (D) are 0.09 Mp and 50° C. respectively.

12. The manufacturing method, as recited in claim 10, wherein said reaction conditions of vacuum level and temperature in the step (D) are 0.09 Mp and 50° C. respectively.

13. The manufacturing method, as recited in claim 12, wherein the amount of anhydrous ethanol used in the step (E) is not less than 0.2 times of said creatine or creatine monohydrate.

14. The manufacturing method, as recited in claim 13, wherein the temperature for drying in the step (F) is 50° C.-60° C.

15. A manufacturing method for preparing a creatine hydrochloride, comprising the steps of:
(A) adding a creatine monohydrate and a suitable aqueous solution of hydrochloric acid into a container, wherein the ratio of said creatine monohydrate VS said hydrochloric acid in said suitable aqueous solution is 1:1;
(B) stirring until all said creatine monohydrate is dissolved to obtain a reaction mixture;
(C) adjusting the value of pH of said reaction mixture to 1.0 by hydrochloric acid;
(D) concentrating the reaction liquid to crystallize and separating the crystal product of said creatine hydrochloride from said reaction mixture; and
(E) flushing said crystal product by absolute ethyl alcohol;
wherein the formula of said creatine hydrochloride is 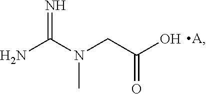
wherein A represents a hydrochloric acid anion.

16. The manufacturing method, as recited in claim 15, further comprising a step (F) of drying said crystal product from the step (E).

17. The manufacturing method, as recited in claim 16, wherein said hydrochloric acid in the step (A) is hydrochloride acid and said creatine hydrochloride is creatine hydrochloride.

18. The manufacturing method, as recited in claim 16, wherein said hydrochloric acid in the step (A) is acetate acid and said creatine hydrochloride is creatine acetate.

19. The manufacturing method, as recited in claim 17, wherein the concentration of said hydrochloride acid is 25-35%.

20. The manufacturing method, as recited in claim 19, wherein the concentration of said hydrochloride acid is 30%.

21. The manufacturing method, as recited in claim 16, wherein the reaction temperature in the step (B) is 25° C.

22. The manufacturing method, as recited in claim 20, wherein the reaction temperature in the step (B) is 25° C.

23. The manufacturing method, as recited in claim 15, wherein the reaction conditions of vacuum level and temperature in the step (D) are 0.08-0.10 Mp and 40° C.-55° C. respectively.

24. The manufacturing method, as recited in claim 22, wherein the reaction conditions of vacuum level and temperature in the step (D) are 0.08-0.10 Mp and 40° C.-55° C. respectively.

25. The manufacturing method, as recited in claim 23, wherein said reaction conditions of vacuum level and temperature in the step (D) are 0.09 Mp and 50° C. respectively.

26. The manufacturing method, as recited in claim 24, wherein said reaction conditions of vacuum level and temperature in the step (D) are 0.09 Mp and 50° C. respectively.

27. The manufacturing method, as recited in claim 26, wherein the amount of anhydrous ethanol used in the step (E) is not less than 0.2 times of said creatine or creatine monohydrate.

28. The manufacturing method, as recited in claim 27, wherein the temperature for drying in the step (F) is 50° C. -60° C.
